(19) United States
(12) Patent Application Publication
(10) Pub. No.: US 2008/0140076 A1
(43) Pub. Date: Jun. 12, 2008

(54) Dynamic stabilization connecting member with slitted segment and surrounding external elastomer

(76) Inventor: Roger P. Jackson, Prairie Village, KS (US)

Correspondence Address: LAW OFFICE OF JOHN C. MCMAHON, P.O. BOX 30069, KANSAS CITY, MO, 64112, US

(21) Appl. No.: 12/069,577

(22) Filed: Feb. 11, 2008

Related U.S. Application Data
(63) Continuation-in-part of application No. 11/888,612, filed on Aug. 1, 2007.
Application No. 11/888,612 is a continuation-in-part of application No. 11/522,503, filed on Sep. 14, 2006.
(60) Provisional application No. 60/900,816, filed on Feb. 12, 2007.
(60) Provisional application No. 60/997,079, filed on Oct. 1, 2007.
(60) Provisional application No. 60/850,464, filed on Oct. 10, 2006.
(60) Provisional application No. 60/722,300, filed on Sep. 30, 2005.
(60) Provisional application No. 60/725,445, filed on Oct. 11, 2005.
(60) Provisional application No. 60/728,912, filed on Oct. 21, 2005.
(60) Provisional application No. 60/736,112, filed on Nov. 10, 2005.
(60) Provisional application No. 60/832,644, filed on Jul. 21, 2006.

(51) Int. Cl. A61B 17/68

(52) U.S. Cl. 606/60

(57) ABSTRACT

A dynamic fixation medical implant having at least two bone anchors includes a longitudinal connecting member assembly having an elongate core that includes the following integral features: a stop plate; a slitted segment; and a threaded segment or a second stop plate. The assembly may further includes a spacer and a nut. The spacer surrounds the slitted segment and the nut is rotatingly mated with the threaded segment. The nut abuts and compresses the spacer against the stop plate and places distractive tension on the slitted segment. Alternative embodiments include a molded spacer cooperating with a neutral, tensioned or bent slitted segment and in some embodiments cooperating with a cable or elastic band.

### CROSS REFERENCE TO RELATED APPLICATIONS

This application claims the benefit of U.S. Provisional Application No. 60/900,816 filed Feb. 12, 2007 and this application claims the benefit of U.S. Provisional Application No. 60/997,079 filed Oct. 1, 2007, both of which are incorporated by reference herein. This application is also a continuation-in-part of U.S. patent application Ser. No. 11/888,612 filed Aug. 1, 2007 that claims the benefit of U.S. Provisional Application No. 60/850,464 filed Oct. 10, 2006, the disclosures of which are incorporated by reference herein. The Ser. No. 11/888,612 application is also a continuation-in-part of U.S. patent application Ser. No. 11/522,503, filed Sep. 14, 2006 that claims the benefit of U.S. Provisional Application Nos. 60/722,300, filed Sep. 30, 2005; 60/725,445, filed Oct. 11, 2005; 60/728,912, filed Oct. 21, 2005; 60/736,112, filed Nov. 10, 2005, and 60/832,644, filed Jul. 21, 2006; the disclosures all of which are incorporated by reference herein.

### BACKGROUND OF THE INVENTION

The present invention is directed to dynamic fixation assemblies for use in bone surgery, particularly spinal surgery, and in particular to longitudinal connecting members and cooperating bone anchors or fasteners for such assemblies, the connecting members being attached to at least two bone anchors.

Historically, it has been common to fuse adjacent vertebrae that are placed in fixed relation by the installation therealong of bone screws or other bone anchors and cooperating longitudinal connecting members or other elongate members. Fusion results in the permanent immobilization of one or more of the intervertebral joints. Because the anchoring of bone screws, hooks and other types of anchors directly to a vertebra can result in significant forces being placed on the vertebra, and such forces may ultimately result in the loosening of the bone screw or other anchor from the vertebra, fusion allows for the growth and development of a bone counterpart to the longitudinal connecting member that can maintain the spine in the desired position even if the implants ultimately fail or are removed. Because fusion has been a desired component of spinal stabilization procedures, longitudinal connecting members have been designed that are of a material, size and shape to largely resist flexure, extension, torsion, distraction and compression, and thus substantially immobilize the portion of the spine that is to be fused. Thus, longitudinal connecting members are typically uniform along an entire length thereof, and usually made from a single or integral piece of material having a uniform diameter or width of a size to provide substantially rigid support in all planes.

An alternative to fusion, which immobilizes at least a portion of the spine, and the use of more rigid longitudinal connecting members or other rigid structure has been a “soft” or “dynamic” stabilization approach in which a flexible loop-, S—, C- or U-shaped member or a coil-like and/or a spring-like member is utilized as an elastic longitudinal connecting member fixed between a pair of pedicle screws in an attempt to create, as much as possible, a normal loading pattern between the vertebrae in flexion, extension, distraction, compression, side bending and torsion. Another type of soft or dynamic system known in the art includes bone anchors connected by flexible cords or strands, typically made from a plastic material. Such a cord or strand may be threaded through cannulated spacers that are disposed between adjacent bone anchors when such a cord or strand is implanted, tensioned and attached to the bone anchors. The spacers typically span the distance between bone anchors, providing limits on the bending movement of the cord or strand and thus strengthening and supporting the overall system. Such cord or strand-type systems require specialized bone anchors and tooling for tensioning and holding the chord or strand in the bone anchors. Although flexible, the cords or strands utilized in such systems do not allow for elastic distraction of the system once implanted because the cord or strand must be stretched or pulled to maximum tension in order to provide a stable, supportive system.

The complex dynamic conditions associated with spinal movement create challenges for the design of elongate elastic longitudinal connecting members that exhibit an adequate fatigue strength to provide stabilization and protected motion of the spine, without fusion, and that allow for some natural movement of the portion of the spine being reinforced and supported by the elongate elastic or flexible connecting member. A further challenge are situations in which a portion or length of the spine requires a more rigid stabilization, possibly including fusion, while another portion or length may be better supported by a more dynamic system that allows for protective movement.

### SUMMARY OF THE INVENTION

Longitudinal connecting member assemblies according to the invention for use between at least two bone anchors provide dynamic, protected motion of the spine and may be extended to provide additional dynamic sections or more rigid support along an adjacent length of the spine, with fusion, if desired. A longitudinal connecting member assembly according to the invention has an inner elongate core or segment, illustrated as a single or discrete substantially solid cylindrical rod-like member, that integrally connects at least first and second bone anchor fixation end portions with at least one stop plate and a slitted segment. In one illustrated embodiment, the assembly includes one stop plate and a fixation segment illustrated as a threaded segment, the slitted segment being disposed between the plate and the fixation segment. The member further includes an outer spacer and a compression/distraction member illustrated as a nut. In such illustrated embodiment, the outer spacer is disposed about the slitted segment and the nut threadably mates with the threaded segment. When threadably attached to the threaded segment, the nut compresses the outer spacer against the stop plate, thereby pulling upon and placing distractive tension on the slitted segment that is integrally attached to both the threaded segment and the plate. In other illustrated embodiments, a slitted segment that is disposed between two stop plates may be pre-tensioned and/or pre-bent and then an elastomer is molded adjacent to or over both stop plates and the slitted segment. In such embodiments, the elastomer and plates cooperate to keep the slitted segment in tension and the spacer located between the plates in compression. Longitudinal connecting member assemblies of the invention may be neutral, pre-tensioned and/or pre-bent prior to being operatively attached to at least a pair of bone anchors along a patient's spine. In pre-tensioned embodiments, the tensioned slitted segment and the compressed spacer cooperate dynamically, both features having some flexibility in bending also, with the outer or external elastic spacer protecting and limiting flexing movement of the inner slitted segment. The outer spacer also protects against tissue growth into the slitted segment. The spacer may include one or more grooves to aid in compression upon installation between the plate and the nut or when over-molded. Embodiments according to the present invention advantageously allow for axial distraction and compression of the connecting member assembly, thus, for example, providing shock absorption. While a threaded nut is shown for pretensioning in one of the embodiments, other structures can be used, such as slip-on clips.

Another aspect of the invention includes providing a longitudinal connecting member that includes an inner core having a helical slit, at least one stop plate integral with the inner core and an elastic spacer surrounding the helical slit, preferably molded there-around, the stop plate and the spacer each extending in at least one direction lateral to the core an amount sufficient for the stop plate and the spacer to cooperate to substantially resist bending moment of the core. Embodiments include, but are not limited to cylindrical as well as an elongate, irregular or non-uniform plate and spacer combinations that extend a sufficient distance away from the core in at least one direction so as to advantageously participate in resisting a slitted core bending moment as compared to sheathed connecting members known in the art that may stiffen a flexible area, particularly with respect to compression, but are otherwise disposed in or near the core and are closely bound or sheathed to the core and of a thickness to substantially bend along with a flexible core. For example, some known connecting members include thin tubular sheaths or even hour-glass shaped sheaths that bend or become concave at a location of bending of an adjacent core rather than bulging outwardly and resisting bending moment such as certain illustrated embodiments of the present invention.

A variety of embodiments according to the invention are possible. For example, the inner elongate core may extend between three or more bone anchors with some or all of the sections that are located between bone anchors having a slit and cooperating spacer. Alternatively, some of the sections may be of a more rigid construction and not include slits and spacers.

### OBJECTS AND ADVANTAGES OF THE INVENTION

An object of the invention is to provide dynamic medical implant stabilization assemblies having longitudinal connecting members that include an inner core having a flexible portion that allows for some protected bending, torsion, compression and distraction of the assembly. Another object of the invention is to provide such an assembly wherein the flexible portion may be pre-tensioned and/or pre-bent while a cooperating portion is pre-compressed. A further object of the invention is to provide dynamic medical implant longitudinal connecting members that may be utilized with a variety of bone screws, hooks and other bone anchors. Another object of the invention is to provide a more rigid or solid connecting member portion or segment, if desired, such as a solid rod portion integral to the core having the flexible portion. Additionally, it is an object of the invention to provide a lightweight, reduced volume, low profile assembly including at least two bone anchors and a longitudinal connecting member therebetween. Furthermore, it is an object of the invention to provide apparatus and methods that are easy to use and especially adapted for the intended use thereof and wherein the apparatus are comparatively inexpensive to make and suitable for use.

Other objects and advantages of this invention will become apparent from the following description taken in conjunction with the accompanying drawings wherein are set forth, by way of illustration and example, certain embodiments of this invention.

The drawings constitute a part of this specification and include exemplary embodiments of the present invention and illustrate various objects and features thereof.

### BRIEF DESCRIPTION OF THE DRAWINGS

FIG. 1 is an enlarged front elevational view of a dynamic fixation connecting member assembly according to the invention including an inner elongate core, an outer spacer and a compression/distraction nut.

FIG. 2 is an enlarged exploded front elevational view of the assembly of FIG. 1.

FIG. 3 is an enlarged and exploded perspective view of the assembly of FIG. 1.

FIG. 4 is a cross-sectional view taken along the line 4-4 of FIG. 2.

FIG. 5 is an enlarged cross-sectional view taken along the line 5-5 of FIG. 2.

FIG. 6 is a perspective and partially exploded view of the assembly of FIG. 1 shown with a pair of bone screws and cooperating closure tops.

FIG. 7 is an enlarged front elevational view of a second embodiment of a dynamic fixation connecting member assembly according to the invention.

FIG. 8 is a cross-sectional view taken along the line 8-8 of FIG. 7.

FIG. 9 is an enlarged and partial perspective view of the assembly of FIG. 7 shown with three bone screws.

FIG. 10 is an enlarged front elevational view of a third embodiment of a dynamic fixation connecting member assembly according to the invention including an inner elongate core, a pair of stop plates and an outer over-molded elastic spacer.

FIG. 11 is a reduced perspective view of the embodiment of FIG. 10 shown before tensioning and molding of the spacer thereon.

FIG. 12 is an enlarged cross-sectional view taken along the line 12-12 of FIG. 10.

FIG. 13 is an enlarged front elevational view of a fourth embodiment of a dynamic fixation connecting member assembly according to the invention including an inner elongate core, a pair of stop plates and an outer over-molded elastic spacer and showing a bone screw in phantom.

FIG. 14 is an enlarged front elevational view, similar to FIG. 13, with portions broken away to show the detail thereof.

FIG. 15 is a cross-sectional view taken along the line 15-15 of FIG. 13.

FIG. 16 is an enlarged top plan view of a fifth dynamic fixation connecting member assembly according to the invention including an integral elongate core member, an outer molded spacer and a pair of connective cables.

FIG. 17 is an enlarged top plan view of the core member of FIG. 16.

FIG. 18 is an enlarged front elevational view of the assembly of FIG. 16 with portions broken away to show the detail thereof.

FIG. 19 is an enlarged perspective view of the assembly of FIG. 16.

FIG. 20 is an enlarged front elevational view of a sixth alternative embodiment of a dynamic fixation connecting member assembly according to the invention with portions broken away to show the detail thereof.

FIG. 21 is an enlarged perspective view of a seventh alternative embodiment of a dynamic fixation connecting member assembly according to the invention.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. It is also noted that any reference to the words top, bottom, up and down, and the like, in this application refers to the alignment shown in the various drawings, as well as the normal connotations applied to such devices, and is not intended to restrict positioning of the connecting member assemblies of the application and cooperating bone anchors in actual use.

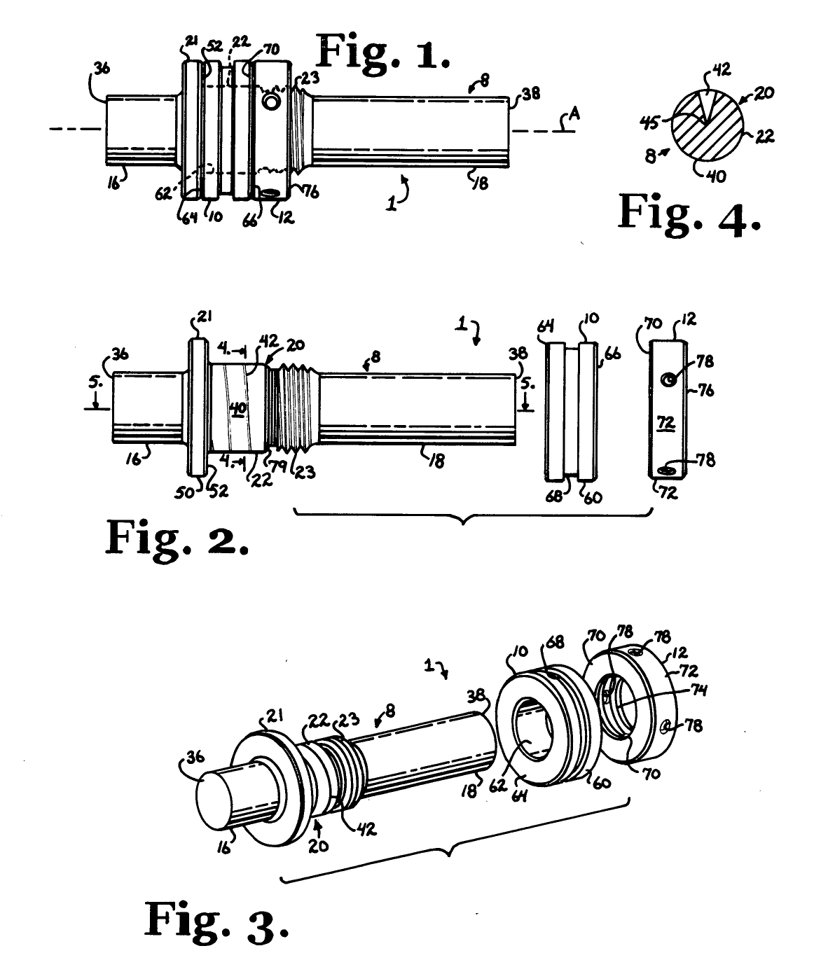

With reference to FIGS. 1-6, the reference numeral 1 generally designates a non-fusion dynamic stabilization longitudinal connecting member assembly according to the present invention. The connecting member assembly 1 includes an inner elongate core or segment, generally 8, an outer sleeve or spacer 10 and a compression/distraction nut 12. The illustrated elongate core 8 is cylindrical and substantially solid, having a central longitudinal axis A. The core 8 further includes bone attachment end portions 16 and 18 and a dynamic segment or mid-portion, generally 20, disposed therebetween. The dynamic mid-portion further includes a stop plate 21, a slitted segment 22 and a threaded segment 23. The inner core 8 is receivable in the outer spacer 10, with the spacer 10 surrounding the slitted segment 22 as will be described more fully below. In the embodiment shown, the inner core 8 is also receivable in the nut 12, an inner thread of the nut 12 mating with the outer threaded segment 23 as will be described more fully below. The dynamic connecting member assembly 1 cooperates with at least a pair of bone anchors, such as the polyaxial bone screws, generally 25 and cooperating closure structures 27 shown in FIG. 6, the assembly 1 being captured and fixed in place at the end portions 16 and 18 by cooperation between the bone screws 25 and the closure structures 27 with the dynamic mid-portion 20 (that is pre-loaded and pre-tensioned with the outer spacer 10 and the nut 12) being disposed between the bone screws 25.

Because the end portions 16 and 18 are substantially solid and cylindrical, the connecting member assembly 1 may be used with a wide variety of bone anchors already available for cooperation with rigid rods including fixed, monoaxial bone screws, hinged bone screws, polyaxial bone screws, and bone hooks and the like, with or without compression inserts, that may in turn cooperate with a variety of closure structures having threads, flanges, or other structure for fixing the closure structure to the bone anchor, and may include other features, for example, break-off tops and inner set screws. It is foreseen that the substantially cylindrical core 8 that has various circular cross-sections may in other embodiments of the invention have other cross-sectional shapes, either along an entire length of the core 8 or portions thereof, including, but not limited to oval, square, rectangular and other curved or polygonal shapes. The bone anchors, closure structures and the connecting member assembly 1 are then operably incorporated in an overall spinal implant system for correcting degenerative conditions, deformities, injuries, or defects to the spinal column of a patient.

The illustrated polyaxial bone screws 25 each include a shank 30 for insertion into a vertebra (not shown), the shank 30 being pivotally attached to an open receiver or head 31. The shank 30 includes a threaded outer surface and may further include a central cannula or through-bore disposed along an axis of rotation of the shank to provide a passage through the shank interior for a length of wire or pin inserted into the vertebra prior to the insertion of the shank 30, the wire or pin providing a guide for insertion of the shank 30 into the vertebra. The receiver 31 has a pair of spaced and generally parallel arms 35 that form an open generally U-shaped channel therebetween that is open at distal ends of the arms 35. The arms 35 each include radially inward or interior surfaces that have a discontinuous guide and advancement structure mateable with cooperating structure on the closure structure 27. The guide and advancement structure may take a variety of forms including a partial helically wound flangeform, a buttress thread, a square thread, a reverse angle thread or other thread like or non-thread like helically and partial helically wound advancement structure for operably guiding under complete and partial rotation and advancing the closure structure 27 downward between the receiver arms 35 and having such a nature as to resist splaying of the arms 35 when the closure 27 is advanced into the U-shaped channel. For example, a flange form on the illustrated closure 27 and cooperating structure on the arms 35 is disclosed in Applicant's U.S. Pat. No. 6,726,689, which is incorporated herein by reference. Slide-in and non-helically wound closure mechanisms can also be used.

The shank 30 and the receiver 31 may be attached in a variety of ways. For example, a spline capture connection as described in U.S. Pat. No. 6,716,214, and incorporated by reference herein, is used for the embodiment disclosed herein. Polyaxial bone screws with other types of capture connections may also be used according to the invention, including but not limited to, threaded connections, frictional connections utilizing frusto-conical or polyhedral capture structures, integral top or downloadable shanks, and the like. Also, as indicated above, polyaxial and other bone screws for use with connecting members of the invention may have bone screw shanks that attach directly to the connecting member core 8 or may include compression members or inserts that cooperate with the bone screw shank, receiver and closure structure to secure the connecting member assembly to the bone screw and/or fix the bone screw shank at a desired angle with respect to the bone screw receiver that holds the longitudinal connecting member assembly. Furthermore, although the closure structure 27 of the present invention is illustrated with the polyaxial bone screw 25 having an open receiver or head 31, it foreseen that a variety of closure structure may be used in conjunction with any type of medical implant having an open or closed head, including monoaxial bone screws, hinged bone screws, hooks and the like used in spinal surgery.

To provide a biologically active interface with the bone, the threaded shank 30 may be coated, perforated, made porous or otherwise treated. The treatment may include, but is not limited to a plasma spray coating or other type of coating of a metal or, for example, a calcium phosphate; or a roughening, perforation or indentation in the shank surface, such as by sputtering, sand blasting or acid etching, that allows for bony ingrowth or ongrowth. Certain metal coatings act as a scaffold for bone ingrowth. Bio-ceramic calcium phosphate coatings include, but are not limited to: alpha-tri-calcium phosphate and beta-tri-calcium phosphate (Ca₃ (PO₄)₂, tetra-calcium phosphate (Ca₄P₂O₉), amorphous calcium phosphate and hydroxyapatite (Ca₁₀(PO₄)₆ (OH)₂). Coating with hydroxyapatite, for example, is desirable as hydroxyapatite is chemically similar to bone with respect to mineral content and has been identified as being bioactive and thus not only supportive of bone ingrowth, but actively taking part in bone bonding.

The longitudinal connecting member assembly 1 illustrated in FIGS. 1-6 is elongate, with the inner core 8 being made from metals and metal alloys, including, but not limited to stainless steel, titanium and titanium alloys, including Nickel titanium (NiTi; commonly referred to by the trade name Nitinol) or other suitable materials, including plastic polymers such as polyetheretherketone (PEEK), ultra-high-molecular weight-polyethylene (UHMWP), polyurethanes and composites. The outer sleeve or spacer 10 may be made of a variety of materials including plastics and composites. The illustrated spacer 10 is made from a plastic, such as a thermoplastic elastomer, for example, polycarbonate-urethane. In order to reduce the production of micro wear debris, that in turn may cause inflammation, it is desirable to make the inner core 8 from a different material than the spacer 10. Additionally or alternatively, in order to result in adequate hardness and low or no wear debris, the spacer 10 inner surfaces and/or cooperating core 8 outer surfaces may be coated with an ultra thin, hard, slick and smooth coating, such as may be obtained from ion bonding techniques and/or other gas or chemical treatments.

Specifically, the illustrated core 8 is a substantially solid, smooth and uniform cylinder or rod having outer cylindrical surfaces of various diameters. It is foreseen that in some embodiments, the core 8 may include a small central lumen along an entire length thereof and opening at each end thereof to allow for threading therethrough and subsequent percutaneous implantation of the member 1. The illustrated core 8 has an end 36 and an opposite end 38, with the solid end portion 16 terminating at the end 32 and the solid end portion 18 terminating at the end 38. The portions 16 and 18 are each sized and shaped to be received in the U-shaped channel formed between the arms 31 of a bone screw 25 with the dynamic mid-portion 20 disposed between cooperating bone screws 25.

With particular reference to FIGS. 1-5, the mid-portion 20 includes the slitted segment 22 disposed between the stop plate 21 and the threaded segment 23. The segment 22, plate 21 and segment 23 are coaxial with the end portions 16 and 18, thus all having an axis A. It is noted however, that in certain embodiments according to the invention, if it is desirable to bend a portion of the core 8 to promote a desired spinal alignment, for example, one or both of the rigid portions 16 and 18 may be pre-bent and/or the slitted portion 22 may also be pre-bent. Also, in other embodiments of the invention, the slitted segment may be disposed between two stop plates and then be pre-tensioned or distracted and (1) a compressed spacer slipped over and around the slitted segment and between the plates; or (2) an elastomer may be over-molded around the pre-tensioned slitted segment or segments, for example, as described in greater detail below with respect to an alternative assembly of the invention, generally 201.

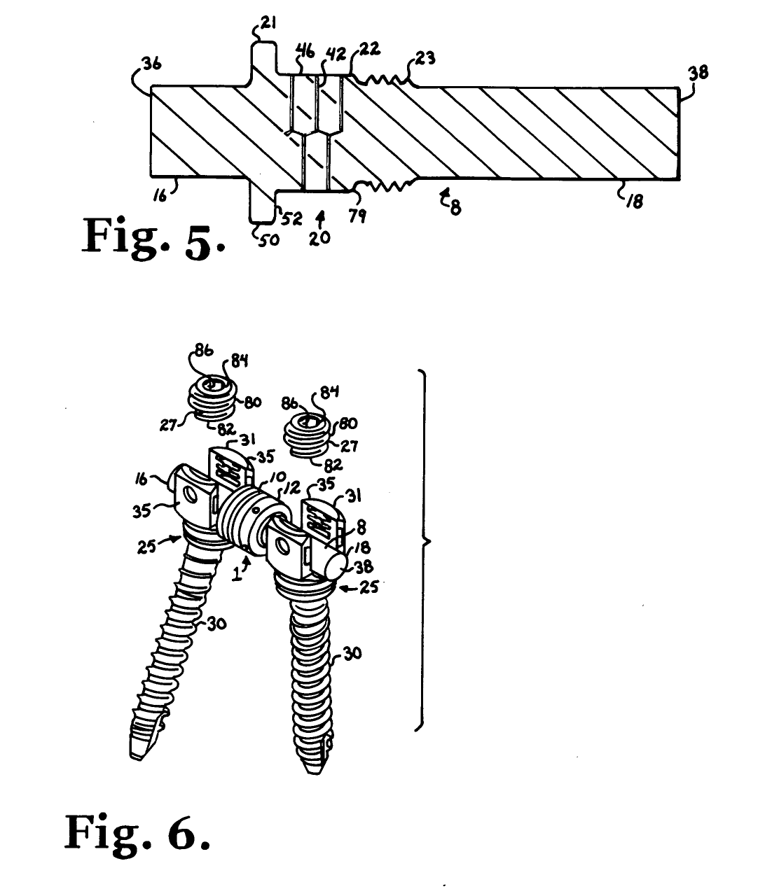

The slitted segment 22 has an outer cylindrical surface 40 of substantially circular cross-section and a helical slit 42 formed therein as best illustrated in FIGS. 4 and 5. However, the slitted segment and other segments or portions of the device could have different cross-sectional shapes. In the illustrated embodiment, a process of forming the helical slit 42 creates an inner, non-linear but substantially central channel 45. The slit 42 runs in a helical pattern along the segment 22 from the plate 21 to the threaded segment 23 and thus the section 22 is expandable and contractible having a spring-like nature. The section 22 provides relief (e.g., shock absorption) and limited movement with respect to flexion, extension, torsion, distraction and compressive forces placed on the assembly 1. Additionally, the section 22 is integral with solid portions or segments of the core 8 at either end thereof, in particular to the plate 21 and the threaded segment 23, which are in turn integral with solid rod portions, thus providing stability and ease in connectability with a wide variety of bone anchors. Furthermore, the slitted segment 22 is of substantially the same or slightly larger diameter as the other solid rod end portions 16 and 18 of the core 8, providing for a non-bulky, low profile connecting member segment.

The solid stop plate 21 includes an outer cylindrical surface 50 that has a diameter greater than a diameter of the slitted segment 22. The plate 21 also has a circular cross-section. The stop plate 21 further includes an annular substantially planar surface 52 that extends from the slitted segment surface 40 to the plate surface 50 and is perpendicular to the axis A. The stop plate 21 is integral with the end portion 16 and the slitted segment 22.

The spacer 10 advantageously cooperates with the core helical slit 42, providing limitation and protection of movement of the core 8 at the slitted segment 22. The spacer 10 also protects patient body tissue from damage that might otherwise occur in the vicinity of the helical slit 42. The spacer 10 is sized and shaped for substantially precise alignment about the section 22 and between the plate surface 52 and the nut 12. Furthermore, as will be discussed in greater detail below, prior to implantation of the assembly 1, the spacer 10 is compressed between the plate 21 and the nut 12 that both compresses the spacer 10 and slightly distracts and tensions the slitted segment 22. Such dynamic tension/compression relationship between the spacer 10 and the slitted section 22 provides further strength and stability to the overall assembly and also allows for the entire connecting member assembly 1 to elongate, if needed, in response to spinal movement. The increased stability and strength of the assembly advantageously allows for use of a smaller, more compact, reduced volume, lower profile longitudinal connecting member assembly 1 and cooperating bone anchors than, for example, flexible cord and spacer type longitudinal connecting member assemblies or coiled traditional spring-like connecting members.

The spacer 10 is substantially cylindrical with an external substantially cylindrical surface 60 that has the same or substantially similar diameter as the diameter of the outer cylindrical surface 50 of the stop plate 21. The spacer is annular and thus further includes an internal substantially cylindrical and smooth inner surface 62. The surface 62 defines a bore with a circular cross section, the bore extending through the spacer 10. Substantially planar opposed end or abutment surfaces 64 and 66 are located on either side of the outer and inner cylindrical surfaces 60 and 62. In the illustrated embodiment, the spacer 10 further includes a compression groove 68. Spacers according to the invention may include one, none or any desired number of grooves 68. The illustrated groove 68 is substantially uniform and circular in cross-section as illustrated in FIGS. 2 and 3, being formed in the external surface 60 and extending radially toward the internal surface 62. The internal surface 62 is of a slightly greater diameter than an outer diameter of the slitted segment surface 40, allowing for axially directed sliding movement of the spacer 10 with respect to the core 8 with the exception of the plate 21. In particular the internal surface 62 is sized to closely but slidingly fit about the segment 22. When the cylindrical core 8 end 38 is inserted in the spacer 10 and the spacer 10 is moved into an ultimate operative position, the spacer 10 completely surrounds the helical slit 42 of the slitted segment 22. When fully assembled and compressed, the spacer surface 64 abuts the stop plate surface 52 and the surface 66 abuts a planar surface 70 of the nut 12 as will be described in greater detail below. It is noted that in addition to dynamic compression and expansion, the spacer 10 limits the bendability of the core 8 and thus provides strength and stability to the assembly 1 and also keeps scar tissue from growing into the core 8 through the helical slit 42, thus eliminating the need for a sheath-like structure to be placed, adhered or otherwise applied to the core 8. The spacer may also include a longitudinal slit or opening so as to be inserted around the slitted segment.

The compression/distraction nut 12 is substantially cylindrical with an external substantially cylindrical surface 72 that has the same or substantially the same diameter as the spacer 10 surface 60. The nut 12 is annular and thus further includes an internal substantially cylindrical threaded surface 74 sized and shaped to mate with the threaded segment 23 under rotation. The inner threaded surface 74 defines a bore with a circular cross section, the bore extending through the nut 12. Substantially planar opposed end or abutment surfaces 70 and 76 are located on either side of the outer and inner cylindrical surfaces 72 and 74. In the illustrated embodiment, the nut 12 further includes four tooling through bores 78 disposed between the cylindrical surfaces 72 and 74. The bores 78 are evenly spaced and provide structure for a holding and driving tool (not shown) used to rotate the nut 12 into mating engagement with the threaded segment 23 and drive the nut 12 against the surface 66 of the spacer 10 thereby compressing the spacer 10. The threaded segment 23 of the core 8 as well as the spacer 10 may be sized and shaped such that abutment and locking of the nut occurs against a shoulder 79 of the slitted segment at a particular location along the threaded segment 23 as illustrated, for example, in FIG. 1, placing the nut 12 in a desired position wherein the spacer 10 is compressed a desired amount and the slitted segment 22 is under a desired amount of tension. In certain embodiments according to the invention, after the nut 12 is positioned on the core 8 and pressing against the spacer 10 with a desired amount of pressure and placing a desired amount of tension on the slitted segment 22, a tool (not shown) may be inserted into one or more of the bores 78 to deform a portion of the thread of the threaded segment 23 and thus lock the nut 12 in a desired position with respect to the threaded segment 23. The nut maybe a hex nut or the like.

The core 8 may be sized and made from such materials as to provide for relatively more or less rigidity along the entire assembly 1, for example with respect to flex or bendability along the assembly 1. Such flexibility therefore may be varied by changing the outer diameter of the various sections of the core 8 and thus likewise changing the inner diametric size of the spacer 10 and the nut 12. Also, since the distance between the bone screw assembly receivers or heads can vary, the core 8 may need to be more or less stiff. The pitch of the helical slit 42 may also be varied to provide a more or less flexible slitted segment 22 and the shock absorption desired. For example, it is noted that increasing the pitch (i.e., forming a more acute angle between the slant of the slit 42 with respect to the axis A) results in a stiffer assembly with respect to bending and axial displacements. Furthermore, a benefit of increasing pitch is a lessening of impact loading between the surfaces defining the helical slit 42, thus dampening the jolts of an impact and improving shock absorption.

With reference to FIG. 6, the closure structure 27 can be any of a variety of different types of closure structures for use in conjunction with the present invention with suitable mating structure on the interior surface of the upstanding arms 35 of the receiver 31. The illustrated closure structure 27 is rotatable between the spaced arms 35, but could be a slide-in closure structure or a partial twist-in closure structure. As described above, the illustrated closure structure 27 is substantially cylindrical and includes an outer helically wound guide and advancement structure in the form of a flange form 80 that operably joins with the guide and advancement structure disposed on the interior of the arms 35. The illustrated closure structure 27 includes a lower or bottom surface 82 that is substantially planar and may include a point and/or a rim protruding therefrom for engaging the core 8 outer cylindrical surface at the non-slitted end portion 16 or 18. The closure may also have a lower separate saddle part. The closure structure 27 has a top surface 84 with an internal drive feature 86, that may be, for example, a star-shaped drive aperture sold under the trademark TORX. A driving tool (not shown) sized and shaped for engagement with the internal drive feature 86 is used for both rotatable engagement and, if needed, disengagement of the closure 27 from the arms 35. The tool engagement structure 86 may take a variety of forms and may include, but is not limited to, a hex shape or other features or apertures, such as slotted, tri-wing, spanner, two or more apertures of various shapes, and the like. It is also foreseen that the closure structure 27 may alternatively include a break-off head designed to allow such a head to break from a base of the closure at a preselected torque, for example, 70 to 140 inch pounds. Such a closure structure would also include a base having an internal drive to be used for closure removal.

In use, at least two bone screws 25 are implanted into vertebrae for use with the longitudinal connecting member assembly 1. Each vertebra may be pre-drilled to minimize stressing the bone. Furthermore, when a cannulated bone screw shank is utilized, each vertebra will have a guide wire or pin (not shown) inserted therein that is shaped for the bone screw cannula of the bone screw shank 30 and provides a guide for the placement and angle of the shank 30 with respect to the cooperating vertebra. A further tap hole may be made and the shank 30 is then driven into the vertebra by rotation of a driving tool (not shown) that engages a driving feature at or near a top of the shank 30. It is foreseen that the screws 25 and the longitudinal connecting member 1 can be inserted in a percutaneous or minimally invasive surgical manner.

With particular reference to FIGS. 1-3, the longitudinal connecting member assembly 1 is assembled by inserting the core 8 at the end 38 into the bore defined by the inner surface 62 of the spacer 10. The spacer 10 is moved toward the end portion 16 until the spacer 10 abuts the stop plate 21 and is disposed about the slitted segment 22, thus covering or encompassing the helical slit 42. The nut 12 is then inserted on the core 8 at the end 38 with the nut surface 70 facing the end 38. The nut 12 is moved toward the spacer 10 and at the section 23 the nut 12 is rotated mating the inner threaded surface 74 with the threaded segment 23. Using a tool (not shown) that extends through a bore or bores 78, the nut 12 is rotated and tightened against the spacer 10 until the nut 12 compresses the spacer 10 against the stop surface 52 and the slitted segment is in distraction or tension. Then a tool (not shown) may be used to deform the threaded segment 23 at the through bores 78 to further lock the nut 12 in place and thus provide an assembly 1 that includes a pre-compressed spacer 10 and cooperating pre-tensioned slitted segment 22 for eventual implantation between the bone screws 25.

With reference to FIG. 6, the pre-tensioned and pre-compressed connecting member assembly 1 is eventually positioned in an open or percutaneous manner in cooperation with the at least two bone screws 25 with the plate 21, spacer 10 and nut 12 disposed between the two bone screws 25 and the end portions 16 and 18 each within the U-shaped channels of the two bone screws 25. A closure structure 27 is then inserted into and advanced between the arms 35 of each of the bone screws 25. The closure structure 27 is rotated, using a tool (not shown) engaged with the inner drive 86 until a selected pressure is reached at which point the core 8 is urged toward, but not completely seated in the u-shaped channels of the bone screws 25. For example, about 80 to about 120 inch pounds pressure may be required for fixing the bone screw shank 30 with respect to the receiver 31 at a desired angle of articulation.

The assembly 1 is thus substantially dynamically loaded and oriented relative to the cooperating vertebra, providing relief (e.g., shock absorption) and protected movement with respect to flexion, extension, distraction and compressive forces placed on the assembly 1 and the two connected bone screws 25. The helical slit 22 and cooperating elastic spacer 10 also allow the core 8 to twist or turn, providing some relief for torsional stresses. The spacer 10, however limits such torsional movement as well as bending movement, providing spinal support. Furthermore, because the spacer 10 is compressed during installation, the spacer and slit combination advantageously allow for some protected extension or distraction of both the core 8 and the spacer 10 as well as compression of the assembly 1.

If removal of the assembly 1 from any of the bone screw assemblies 25 is necessary, or if it is desired to release the assembly 1 at a particular location, disassembly is accomplished by using the driving tool (not shown) with a driving formation cooperating with the closure structure 27 internal drive 86 to rotate and remove the closure structure 27 from the receiver 31. Disassembly is then accomplished in reverse order to the procedure described previously herein for assembly.

Eventually, if the spine requires more rigid support, the connecting member assembly 1 according to the invention may be removed and replaced with another longitudinal connecting member, such as a solid rod, having the same diameter as the inner core 8 end portions 16 and 18, utilizing the same receivers 31 and the same or similar closure structures 27. Alternatively, if less support is eventually required, a less rigid, more flexible assembly, for example, an assembly 1 made of a more flexible material or an assembly 1 having a slit of different pitch, but with end portions having the same diameter as the inner core 8 end portions 16 and 18, may replace the assembly 1, also utilizing the same bone screws 25.

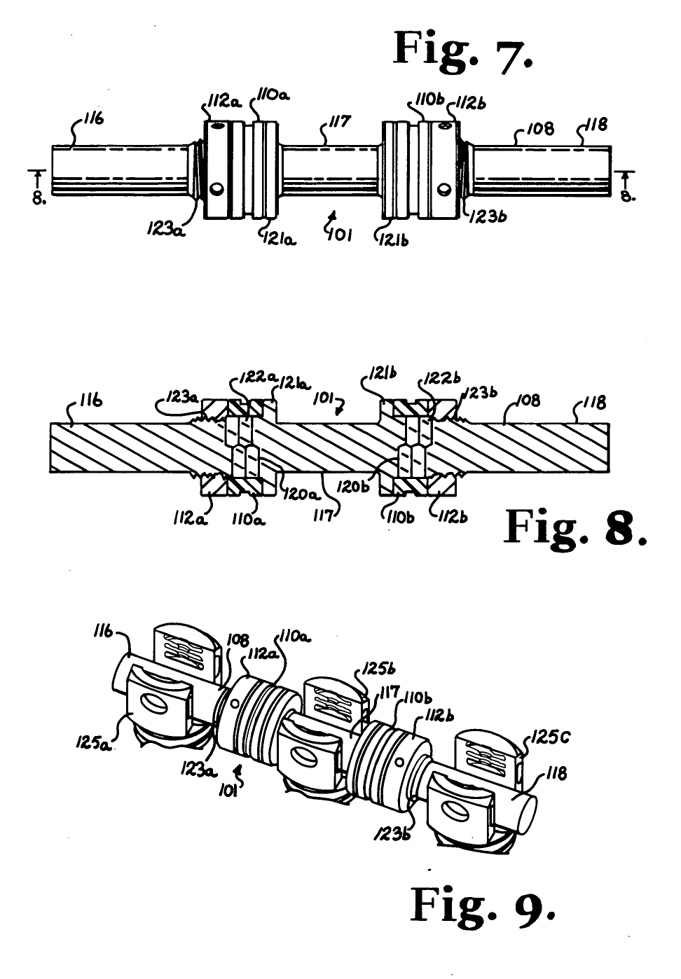

With reference to FIGS. 7-9, an alternative longitudinal connecting member assembly according to the invention, generally 101 includes an inner core 108 cooperating with a pair of outer spacers 110a and 110b and a pair of nuts 112a and 112b. The spacers 110a and lob are the same or substantially similar to the spacer 10 previously described herein with respect to the assembly 1. The nuts 112a and 112b are the same or substantially similar to the nut 12 previously described herein with respect to the assembly 1. The inner core 108 is similar to the core 8 previously described herein with the exception that such core 108 includes a pair of spaced dynamic segments 120a and 120b that are each substantially similar to the dynamic segment 20 previously described herein with respect to the assembly 1. Therefore, each of the dynamic segments 120a and 120b includes respective stop plates 121a and 121b, slitted segments 122a and 122b and threaded segments 123a and 123b that are the same or substantially similar to the stop plate 21, the slitted segment 22 and the threaded segment 23 previously described herein with respect to the assembly 1. Integral with the dynamic segments 120a and 120b are solid rod portions 116, 117 and 118. The solid rod portions 116 terminates at a first end of the core 108 and is adjacent and integral to the dynamic segment 120a. The solid rod portion 117 is integral with and disposed between the dynamic segments 120a and 120b. The solid rod portion 118 is integral with the dynamic segment 120b and terminates at an end of the core 108 opposite of the portion 116 end.

As illustrated in FIG. 9, each of the rod portions 116, 117 and 118 is sized and shaped to cooperate with bone screws 125a, 125b and 125c, respectively. The bone screws 125a, 125b and 125c are the same or similar to the bone screw 25 previously described herein with respect to the assembly 1. Although not shown, each bone screw assembly 125 further includes a closure structure that is the same or similar to the closure structure 27, also previously described herein. As with the assembly 1, the assembly 101 readily cooperates with a wide variety of bone anchors and closures, also as previously described herein.

As indicated above, the connecting member assembly 101 is sized and shaped to attach to at least three bone screw assemblies 125a, b and c, to provide dynamic stabilization between each of the bone screws. It is noted that each of the portions 116, 117 and 118 may also be elongate for cooperating with additional bone screws 125. In use, the assembly 101 is implanted in a manner substantially similar to that previously described herein with respect to the assembly 1.

In the illustrated embodiments, the lengths 16, 18, 116, 117 and 118 have been shown as relatively short in length, each cooperating with a single bone anchor. However, it is foreseen that in certain embodiments according to the invention such solid rod lengths may be longer to accommodate more bone anchors and thus extend along a greater length of the spine. Furthermore, although two dynamic segments are shown in FIGS. 7-9, it is foreseen that dynamic connecting assemblies according to the invention may include a greater number of dynamic segments, each segment equipped with a spacer and some sort of compression member for pressing the spacer against a stop and distracting a slitted segment of the core, each dynamic segment being disposed between cooperating adjacent bone anchors. It is also foreseen that the compression member may be a structure other than a threaded nut, for example the compression member may be slipped on, crimped on, ratcheted or otherwise fixed against the spacer.

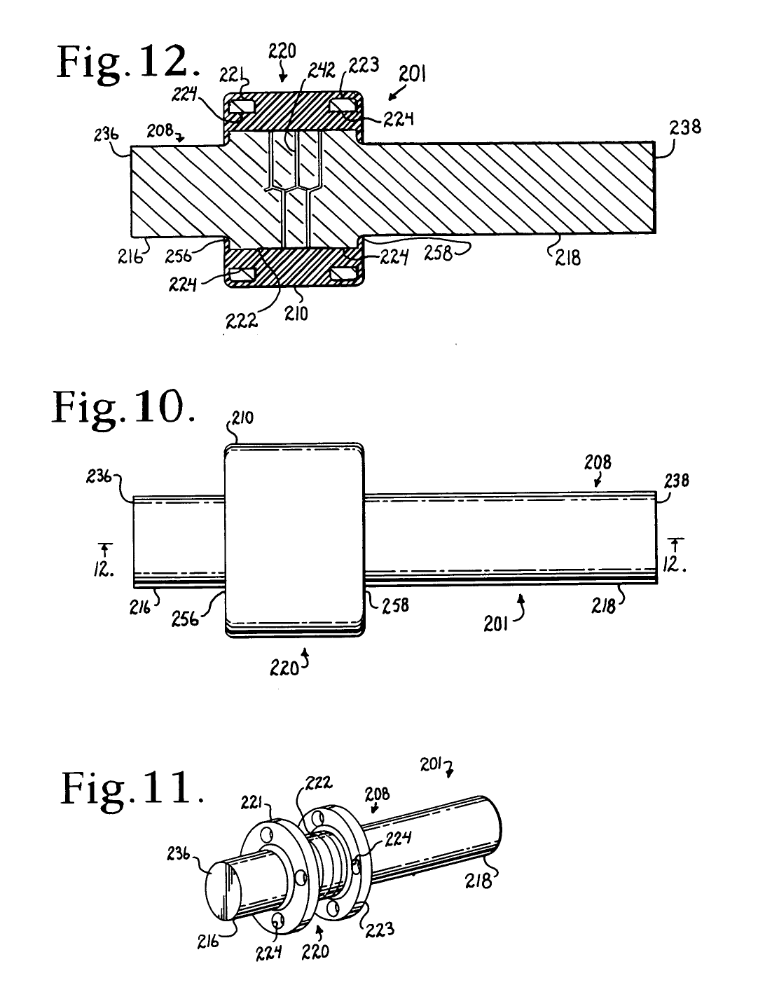

With reference to FIGS. 10-12, a second alternative longitudinal connecting member assembly according to the invention, generally 201 includes an inner core 208 cooperating with an over-molded, external or outer elastic spacer 210. The spacer 210 may be made of materials similar to what was described previously with respect to the spacer 10 of the assembly 1. The elongate core 208 is similar to the core 8 previously described herein with the exception that the core 208 does not include a threaded portion, but rather a second integral plate. Thus the core 208 includes a first end portion 216, a second end portion 218 and a dynamic segment or mid-portion 220 that includes a first stop plate 221, a slitted segment 222 and a second stop plate 223, as well as the over-molded outer or exterior elastic spacer 210. The end portions 216 and 218 are identical or substantially similar to the end portions 16 and 18 of the assembly 1. The stop plates 221 and 223 are substantially similar to the stop plate 21 and the slitted segment 222 is the same or substantially similar to the segment 22 previously described herein with respect to the assembly 1, the slitted segment 222 being disposed between the stop plates 221 and 223. Each of the stop plates 221 and 223 may be solid or include one or up to a plurality of through bores 224 running parallel with the core 208. The illustrated embodiment includes four bores 224 running through each plate 221 and 223.

The solid rod portions 216 and 218 are integral with the dynamic segment 220. The solid rod portion 216 terminates at a first end 236 of the core 208 and is adjacent and integral to the plate 221. The solid rod portion 218 is integral with the plate 223 and terminates at an end 238 of the core 208 opposite the end 236. Similar to the assembly 1 and thus as illustrated in FIG. 6, each of the rod portions 216 and 218 is sized and shaped to cooperate with bone screws 25, for example. As with the assembly 1, the assembly 201 readily cooperates with a wide variety of bone anchors and closures, also as previously described herein. Similar to the assembly 1, the assembly 201 slitted segment 222 is substantially solid with the exception of a helical slit 242 that is the same or substantially similar to the slit 42 previously described herein with respect to the assembly 1.

With particular reference to FIG. 12, the over-molded elastic spacer or portion 210 is molded about and in some cases adhered to the plates 221 and 223, starting at a location 256 adjacent to or adhered to the end portion 216 and ending at a location 258 adjacent to or adhered to the end portion 218. The locations 256 and 258 are spaced from the respective plates 221 and 223 and thus the polymer of the spacer 210 completely surrounds the plates 221 and 223 and the entire slitted segment 222. An outer diameter of the over-molded spacer 210 is greater than outer diameters of the plates 221 and 223. The slitted segment 222 is sheathed or otherwise treated prior to molding to prohibit polymer from entering into the slit 242 during the over-molding process and allow the segment 222 to slidingly engage the spacer 210. As with the assemblies 1 and 101, it is foreseen that according to other embodiments of the invention, the plates 221 and 223, the slitted segment 220 and the over-molded spacer 210 may be of relatively constant cross-section or may have other cross-sectional geometries, including but not limited to oval, square, rectangular and other polygonal shapes. Mixtures of cross-section may be utilized, for example, the plates 221 and 223 and the spacer 210 may be substantially cylindrical while the inner core 208 may be of square or rectangular cross-section.

The longitudinal connector 201 is formed in a factory setting with the inner core 208 being held in a jig or other holding mechanism at the end portions 216 and 218 with the mid-portion 220 being held in tension or distracted as an elastomeric polymer is molded about the slitted segment 222 and the plates 221 and 223. The polymer flows about but not in the slit 242. The polymer also flows through all of the through bores 224, firmly attaching the resulting spacer 210 to the plates 221 and 223. In some cases, the polymer is further firmly adhered to the plates 221 and 223, occurring for example, by chemical bonding or with the aid of an adhesive. The resulting molded spacer 210 surrounds all surfaces of the plates 221 and 223 and the slitted segment 222.

As indicated above, the connecting member assembly 201 is sized and shaped to attach to at least two bone screw assemblies to provide dynamic stabilization between such bone screws. It is noted that each of the portions 216 and 218 may also be elongate for cooperating with additional bone screws 25. In use, the assembly 201 is implanted in a manner substantially similar to that previously described herein with respect to the assembly 1. Furthermore, it is foreseen that dynamic connecting assemblies according to the invention may pre-bent and/or include a greater number of dynamic segments, each segment equipped with an over-molded spacer or a spacer cooperating with some sort of compression member for pressing the spacer against a stop or stops and distracting a slitted segment of the core, each dynamic segment being disposed between cooperating adjacent bone anchors. The connecting assembly 201 is substantially dynamically loaded and oriented relative to the cooperating vertebra, providing relief (e.g., shock absorption) and protected movement with respect to flexion, extension, distraction, compressive, torsion and shear forces placed on the connector 201 and the connected bone screws 25.

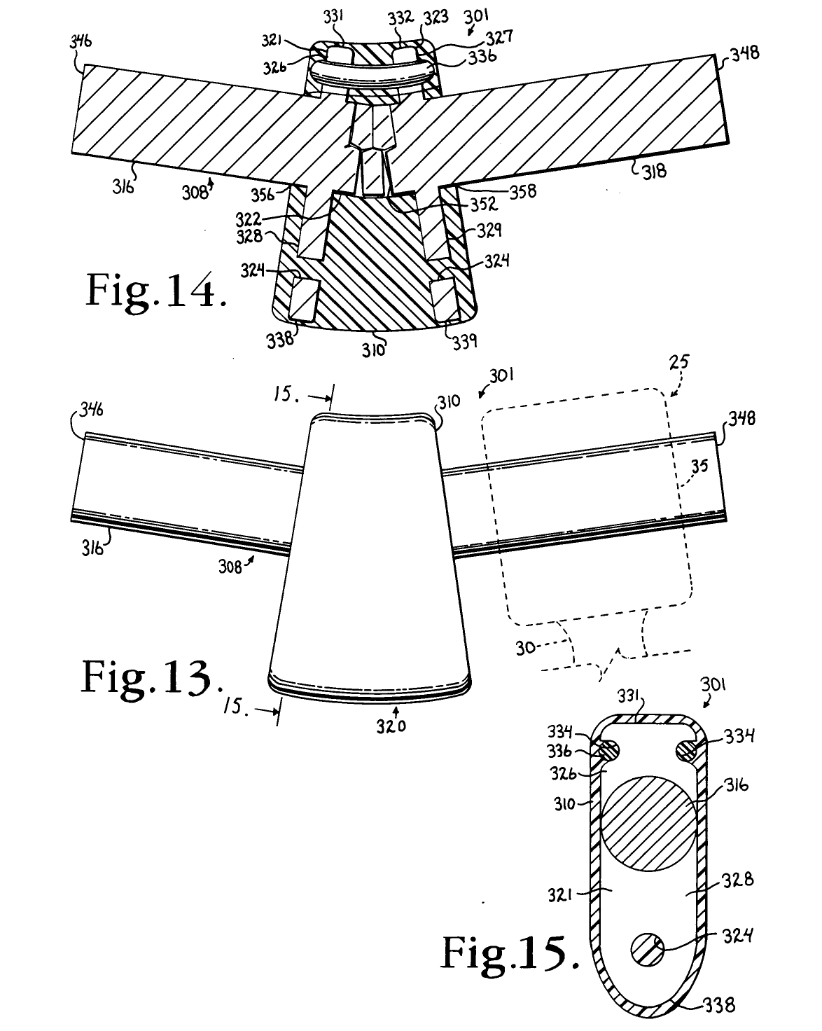

With reference to FIGS. 13-15, a third alternative longitudinal connecting member assembly according to the invention, generally 301 includes an inner core 308 cooperating with an over-molded, external or outer elastic spacer 310. The over-molded spacer 310 may be made of materials similar to what was described previously with respect to the spacer 10 of the assembly 1 and the spacer 210 of the assembly 201, for example. The elongate core 308 is identical or substantially similar to the core 208 previously described herein. Thus the core 308 includes a first end portion 316, a second end portion 318 and a dynamic segment or mid-portion 320 that includes a first stop plate 321, a slitted segment 322 and a second stop plate 323, as well as the over-molded outer or exterior elastic spacer 310. The end portions 316 and 318 are identical or substantially similar to the end portions 216 and 218 of the assembly 201. The stop plates 321 and 323 are substantially similar to the stop plates 221 and 222 with the exception of their shape and location of a through bore 324 that is similar to the bore 224 of the plates 221 and 222. The slitted segment 322 is the same or substantially similar to the segment 222 previously described herein with respect to the assembly 201, the slitted segment 322 being disposed between the stop plates 321 and 323. As with the stop plates 221 and 223, the stop plates 321 and 323 may be solid or include one or up to a plurality of the through bores 324 running alongside the core 308. The illustrated embodiment includes one bore 324 running through each plate 321 and 323. The plates 321 and 323 are identical in size and shape, differing from the plates 221 and 223 in that the plates 321 and 323 have a curved elongate form similar to a surf- or skateboard-shape as compared to the circular cross-sectional shape of the plates 221 and 223. The plates 321 and 323 have respective posterior portions 326 and 327 located substantially on one side of the core 308 and respective anterior portions 328 and 329 located substantially on an opposite side of the core 308 from the portions 326 and 327, the portion 326 being integral with the portion 328 and the portion 327 being integral with the portion 329. The portions 328 and 329 extend a greater length in a direction away from the core 308 than the portions 326 and 327. The portions 326 and 327 are somewhat squared-off in form having substantially flat respective posterior end surfaces 331 and 332. Each of the portions 326 and 327 includes a pair of opposed notches 334 sized and shaped for receiving an elastic band 336 there around, the notches being spaced from the surfaces 331 and 332. The elastic band 336 is made from suitable elastomeric materials, including, but not limited to, synthetic and natural rubbers and blends thereof and other elastic materials previously described herein for the spacer 10 of the assembly 1. One through bore 324 extends through each of the portions 328 and 329 and is located near but spaced from a respective curved anterior surface 338 or 339.

The solid rod portions 316 and 318 are integral with the dynamic segment 320. The solid rod portion 316 terminates at a first end 346 of the core 308 and is adjacent and integral to the plate 321. The solid rod portion 318 is integral with the plate 323 and terminates at an end 348 of the core 308 opposite the end 346. Similar to the assembly 1 and thus as illustrated in FIG. 6, each of the rod portions 316 and 318 is sized and shaped to cooperate with bone screws 25, for example (and as shown in phantom in FIG. 13). As with the assembly 1, the assembly 301 readily cooperates with a wide variety of bone anchors and closures, also as previously described herein. Similar to the assembly 1, the assembly 301 slitted segment 322 is substantially solid with the exception of a helical slit 352 that is the same or substantially similar to the slit 42 previously described herein with respect to the assembly 1.

With particular reference to FIGS. 14 and 15, the over-molded elastic spacer or portion 310 is molded about and in some cases adhered to the plates 321 and 323, starting at a location 356 adjacent to or adhered to the end portion 316 and ending at a location 358 adjacent to or adhered to the end portion 318. The locations 356 and 358 are spaced from the respective plates 321 and 323 and thus the polymer of the spacer 310 completely surrounds the plates 321 and 323 and the entire slitted segment 322. As is best shown in FIG. 15, an outer peripheral surface of the over-molded spacer 310 is greater than outer peripheries of the plates 321 and 323 at every location along the surfaces of the plates 321 and 323. The slitted segment 322 is sheathed or otherwise treated prior to molding to prohibit polymer from entering into the slit 352 during the over-molding process and allow the segment 322 to slidingly engage the spacer 310.

The longitudinal connector 301 is formed in a factory setting with the inner core 308 being held in a jig or other holding mechanism at the end portions 316 and 318 with the mid-portion 320 being held in a bent and at least partially tensioned orientation as shown in FIGS. 13 and 14 as the band 336 is placed about both the plates 321 and 323 at the notches 334. As the elastic band 336 holds or maintains the core 308 in the desired bent orientation, an elastomeric polymer is molded about the slitted segment 322, the plates 321 and 323 and the band 336. The polymer flows about but not into the slit 352. The polymer also flows through the through bores 324, firmly attaching the resulting trapezoidal shaped spacer 310 to the plates 321 and 323. In some cases, the polymer is further firmly adhered to the plates 321 and 323, occurring for example, by chemical bonding or with the aid of an adhesive. The resulting molded spacer 310 surrounds all surfaces of the plates 321 and 323 and the slitted segment 322 and about the elastic band 336.

As indicated above, the connecting member assembly 301 is sized and shaped to attach to at least two bone screw assemblies to provide dynamic stabilization between such bone screws. The surf-board shape of the plates 321 and 323 and cooperating molded spacer 310 advantageously provide a transfer of an operative axis of translation of the resulting medical implant assembly from a posterior to an anterior position (for example, anterior of a facet joint, guarding against overload of such facet in compression). It is noted that each of the portions 316 and 318 may also be elongate for cooperating with additional bone screws 25. In use, the assembly 301 is implanted in a manner similar to that previously described herein with respect to the assembly 1 and in an orientation as generally shown by the bone screw 25 shown in phantom in FIG. 13, with the wider and longer portion of the spacer 320 (and the plate surfaces 338 and 339) being directed anteriorly. Furthermore, it is foreseen that other portions of the assembly 301 may be pre-bent and/or include a greater number of dynamic segments (straight or pre-bent), each segment equipped with an over-molded spacer or a spacer cooperating with some sort of compression member for pressing the spacer against a stop or stops and distracting a slitted segment of the core, each dynamic segment being disposed between cooperating adjacent bone anchors. The connecting assembly 301 is substantially dynamically loaded and oriented relative to the cooperating vertebra, providing relief (e.g., shock absorption) and protected movement with respect to flexion, extension, distraction, compressive, torsion and shear forces placed on the connector 301 and the connected bone screws 25.

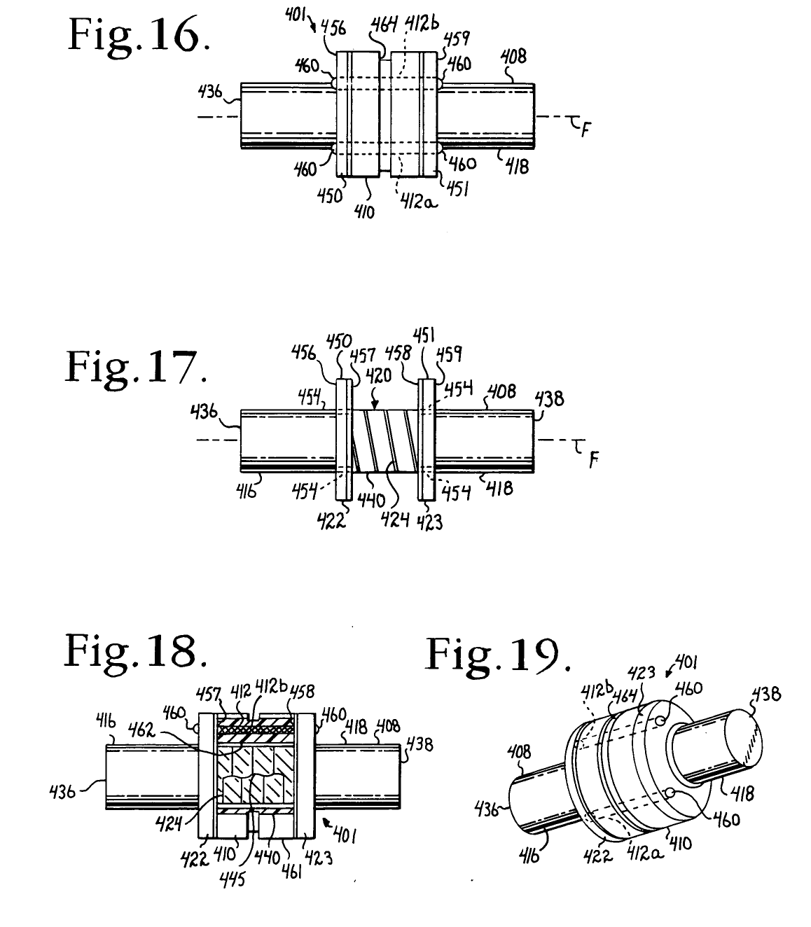

With reference to FIGS. 16-19, the reference numeral 401 generally designates a fourth alternative non-fusion dynamic stabilization longitudinal connecting member assembly according to the present invention. The connecting member assembly 401 includes an elongate core member or segment, generally 408, an outer sleeve or spacer 410 and at least one and up to a plurality of connective cables, generally 412. The core 408 is substantially similar to the cores 8, 108, 208 and 308 previously described herein. The molded spacer 410 is substantially similar to the molded spacers 210 and 310 previously described herein. The illustrated elongate core 408 is cylindrical and substantially solid, having a central longitudinal axis F and of a variety of circular cross-sections taken perpendicular to the axis F. However, it is noted that the core may be of a variety of cross-sectional shapes (taken perpendicular to the axis F), including but not limited to non-circular, such as oval, rectangular, square and other polygonal and curved shapes. With particular reference to FIGS. 17 and 18, the core member 408 further includes bone attachment end portions 416 and 418 and a dynamic segment or mid-portion, generally 420, disposed therebetween. At either end of the mid-portion 420 are integral or fixed rigid abutment or stop plates 422 and 423 with the mid-portion 420 including a helical slit 424. The spacer 410 is molded about the mid-portion 420 in a manner so as not to allow any of the spacer 410 material to flow into the slit 424. The cable or cables 412 that are further identified in the embodiment disclosed in FIGS. 16-19 as cables 412a and 412b are attached to the plates 422 and 423 prior to molding of the spacer 410 therebetween. The dynamic connecting member assembly 401 cooperates with at least a pair of bone anchors, such as the polyaxial bone screws, generally 25 and cooperating closure structures 27 previously described herein, the assembly 401 being captured and fixed in place at the end portions 416 and 418 by cooperation between the bone screws 25 and the closure structures 27 with the dynamic mid-portion 420 (that may be pre-bent or pre-tensioned) and the cooperating outer spacer 410 being disposed between the bone screws 25.

Because the illustrated end portions 416 and 418 are substantially solid and cylindrical, the connecting member assembly 401 may be used with a wide variety of bone anchors already available for cooperation with rigid rods including fixed, monoaxial bone screws, hinged bone screws, polyaxial bone screws, and bone hooks and the like, with or without compression inserts, that may in turn cooperate with a variety of closure structures having threads, flanges, or other structure for fixing the closure structure to the bone anchor, and may include other features, for example, break-off tops and inner set screws. It is foreseen that the substantially cylindrical core 408 that has various circular cross-sections may in other embodiments of the invention have other cross-sectional shapes, either along an entire length of the core 408 or portions thereof, including, but not limited to oval, square, rectangular and other curved or polygonal shapes. The bone anchors, closure structures and the connecting member assembly 401 are then operably incorporated in an overall spinal implant system for correcting degenerative conditions, deformities, injuries, or defects to the spinal column of a patient.

The longitudinal connecting member assembly 401 illustrated in FIGS. 16-19 is elongate, with the section 416, the plate 422, the section 420, the section 423 and the section 418 being integral, the core 408 preferably being made from metal, metal alloys or other suitable materials, including plastic polymers such as polyetheretherketone (PEEK), ultra-high-molecular weight-polyethylene (UHMWP), polyurethanes and composites. The spacer 410 may be made of a variety of materials including plastics and composites. The illustrated spacer 410 is a molded thermoplastic elastomer, for example, polyurethane or a polyurethane blend; however, any suitable polymer material may be used.

Specifically, the illustrated core 408 is a substantially solid, smooth and uniform cylinder or rod having outer cylindrical surfaces of various diameters. It is foreseen that in some embodiments, the core 408 may include a small central lumen along an entire length thereof and opening at each end thereof to allow for threading therethrough and subsequent percutaneous implantation of the member 401. The illustrated core member 408 has an end 436 and an opposite end 438, with the solid end portion 416 terminating at the end 436 and the solid end portion 418 terminating at the end 438. The portions 416 and 418 are each sized and shaped to be received in the U-shaped channel formed between the arms 435 of a bone screw 25 with the dynamic mid-portion 420 disposed between cooperating bone screws 25.

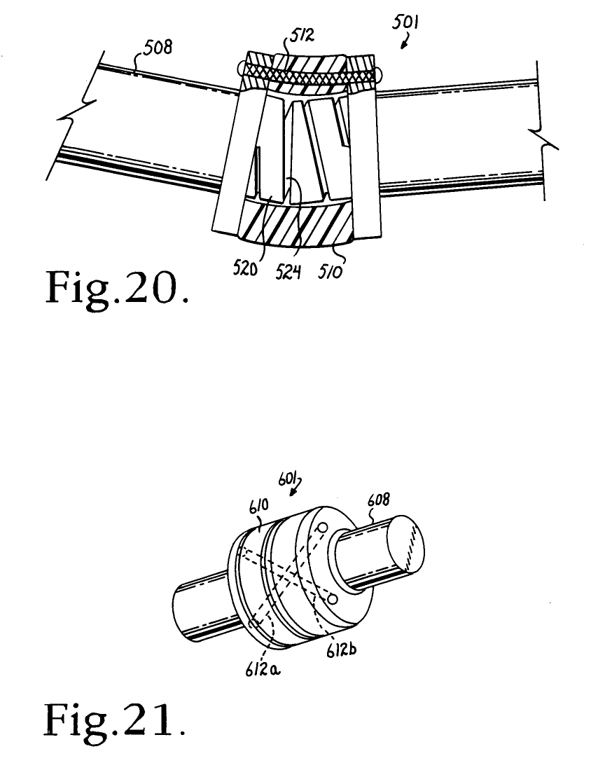

With particular reference to FIGS. 17 and 18, the mid-portion 420 includes the slit 424 that is disposed between the stop plate 422 and the stop plate 423. The portion 420 and plates 422 and 423 are coaxial with the end portions 416 and 418, thus all aligned along the axis F. It is noted however, that in certain embodiments according to the invention, the portion 420 may be bent as shown in FIG. 20. Also, in certain embodiments it may be desirable to bend a more rigid portion of the core 408 to promote a desired spinal alignment, for example, the portion 418 may be bent.

The slitted portion 420 has an outer cylindrical surface 440 of substantially circular cross-section with the helical slit 424 formed therein. In the illustrated embodiment, a process of forming the helical slit 424 creates an inner, non-linear but substantially central channel 445. The slit 424 runs in a helical pattern along the portion 420 from the plate 422 to the plate 423 and thus the section or portion 420 is expandable and contractible having a spring-like nature. The portion 420 provides relief (e.g., shock absorption) and limited movement with respect to flexion, extension, torsion, distraction and compressive forces placed on the assembly 401. As previously described above, the portion 420 is integral with the plates 422 and 423 at either end thereof, which are in turn integral with solid rod portions, thus providing stability and ease in connectability with a wide variety of bone anchors. Furthermore, the slitted portion 420 is of substantially the same or slightly larger diameter than the other solid rod end portions 416 and 418 of the core 408, providing for a non-bulky, low profile connecting member segment. It is foreseen that in certain embodiments according to the invention, the slitted portion 420 may be of a smaller diameter than the rod portions 416 and 418 and the plates 422 and 423 may be of slightly larger diameter than the rod portions 416 and 418. In other embodiments it is foreseen that the plates 422 and 423 may be eliminated if the slitted portion 420 is smaller in diameter than the rod portions 416 and 418. In such embodiments, the longitudinal connecting member of the invention could have a uniform outer diameter along the entire length thereof once the spacer component is molded thereon.

In the embodiments shown, the solid plates 422 and 423 each include an outer cylindrical surface 450 and 451, respectively, having a diameter greater than a diameter of the slitted segment 420. The plates 422 and 423 also each have a circular cross-section; however, it is foreseen that rectangular or other cross-sectional shapes could be used. Each plate has apertures or grooves 454 running therethrough sized and shaped to receive one of the cables 412 therethrough. The stop plate 422 includes a pair of opposed substantially planar end surfaces 456 and 457 and the stop plate 423 includes a pair of opposed substantially planar end abutment surfaces 458 and 459. The plate surfaces 457 and 458 face one another and the slit 424 is located therebetween. The grooves or apertures 454 run between the surfaces 456 and 457 and also between the surfaces 458 and 459. In the illustrated embodiment, with respect to the axis F, on each respective plate 422 or 423, the two grooves or apertures 454 are located at about 120 degrees from one another. In operation, the apertures 454 are positioned so as to position the two cables 412 at a substantial equal distance from a line directed squarely toward the spinal column with both of the cables 412 located posterior of the core 408. Stated in another way, the apertures 454 are located so as to position the pair of attached cables 412 at ten o'clock and two o'clock with twelve o'clock being a location furthest away from the spine or most posterior to the spine and six o'clock being a location being closest to or most anterior with respect to the spine.

The cables 412 are threaded through apertures 454 and may be fastened or knotted at surfaces 456 and 459, such as illustrated by four pins 460, two at the surface 456 and two at the surface 459, the pins 460 being fixed to either end of each cable 412a and 412b and sized and shaped to be larger than the apertures 454 and thus not receivable therethrough. Each cable 412 extends between the plates 422 and 423 and functions as a check, limitation or restraint with respect to certain bending angles and/or rotation, as will be described in greater detail below. Because the cables 412 are attached to the assembly 401 and then encased in the molded spacer 410, it is foreseen that according to the invention the apertures 454 may be grooves that extend to the surfaces 450 and 451 and the cables 412 equipped with attached or integral end pegs or pins may be received into the apertures 454 at the surfaces 450 and 451 rather than threaded through a circular aperture as shown. Thereafter, the molded material of the spacer 410 keeps the cables 412 and cooperating pins in place on the assembly 401. The cables 412 may take a variety of forms including but not limited to, cords, threads, strings, bands, fibers of single or multiple strands, including twisted or plated materials. The cables 412 may be made from a variety of material including but not limited to metals, metal alloys (e.g., stainless steel or titanium cables), and polyester fibers.

The spacer 410 advantageously cooperates with the core helical slit 424, also cooperating with the cable or cables 412 to provide limitation and protection of movement of the core member 408 at the slitted portion 420. The spacer 410 helps keep scar tissue from growing into the slit and also protects patient body tissue from damage that might otherwise occur in the vicinity of the helical slit 424. The spacer 410 is sized and shaped for substantially precise alignment about the section 420 and between the plate surfaces 457 and 458 of respective plates 422 and 423. Furthermore, as will be discussed in greater detail below, prior to molding, the section 420 may be angulated and/or tensioned or expanded, resulting in the spacer 410 being in a pre-compressed state when implanted with the portion 420 being pre-tensioned. Such dynamic tension/compression relationship between the spacer 410 and the slitted portion 420 provides further strength and stability to the overall assembly and also allows for the entire connecting member assembly 401 to elongate, if needed, in response to spinal movement. The increased stability and strength of the assembly 401 advantageously allows for use of a smaller, more compact, reduced volume, lower profile longitudinal connecting member assembly 401 and cooperating bone anchors than, for example, flexible cord and spacer type longitudinal connecting member assemblies or coiled traditional spring-like connecting members.

The molded spacer 410 is fabricated about the portion 420 from a molded elastomer, as will be described more fully below, in the presence of the segments 416 and 418, with molded plastic flowing about the cables 412a and 412b but not within the slit 424. Thereafter, the elastomer surrounds and may adhere to the cables 412. The elastomer engages and may adhere to the surfaces 457 and 458. The formed elastomer is substantially cylindrical with an external substantially cylindrical surface 461 that has the same or substantially similar diameter as the diameter of the outer cylindrical surfaces 450 and 451 of the respective stop plates 422 and 423. It is foreseen that in some embodiments, the spacer may be molded to be of square, rectangular or other outer and inner cross-sections including curved or polygonal shapes. The spacer further includes an internal substantially cylindrical and smooth inner surface 462 spaced from the surface 440 of the portion 420. The surface 462 defines a bore with a circular cross section, the bore extending through the spacer 410. In the illustrated embodiment, the spacer 410 further includes a compression groove 464. Spacers according to the invention may include one, none or any desired number of grooves 464. The illustrated groove 464 is substantially uniform and circular in cross-section as illustrated in FIGS. 16 and 18, being formed in the external surface 461 and extending radially toward the internal surface 462. During the molding process a sleeve or other material (not shown) is placed on the surface 440 of the portion 420 so that the internal surface 462 is of a slightly greater diameter than an outer diameter of the slitted segment surface 440, allowing for axially directed sliding movement of the spacer 410 with respect to the portion 420.

The core member 408 may be sized and made from such materials as to provide for relatively more or less rigidity along the entire assembly 401, for example with respect to flex or bendability along the assembly 401. Such flexibility therefore may be varied by changing the outer diameter or width of the various sections of the core 408 and thus likewise changing the inner diametric size or width of the spacer 410. Also, since the distance between the bone screw assembly receivers or heads can vary, the core member 408 may need to be more or less stiff. The pitch of the helical slit 424 may also be varied to provide a more or less flexible slitted portion 420 and the shock absorption desired. For example, it is noted that increasing the pitch (i.e., forming a more acute angle between the slant of the slit 424 with respect to the axis F) results in a stiffer assembly with respect to bending and axial displacements. Furthermore, a benefit of increasing pitch is a lessening of impact loading between the surfaces defining the helical slit 424, thus dampening the jolts of an impact and improving shock absorption.

With reference to FIGS. 16-19, the longitudinal connecting member assembly 401 is assembled by first connecting each of the cables 412a and 412b to the plates 450 and 451 followed by fabricating the spacer 410. Specifically, the core member 408 is placed in a jig or other holding mechanism that frictionally engages and holds the sections 416 and 418, for example, and the spacer 410 is molded about the portion 420 to form a substantially solid cylinder between the plate surface 457 of the plate 422 and the surface 458 of the plate 423, with the cables 412a and 412b located between the plates 422 and 423 and a sheath, such as a gel, celluloid wrapper or other substance placed about the surface 440 of the slitted portion 420 so that the plastic substance forming the spacer 410 does not flow into the slit 424. The cables 412 are typically neutral (slack) during the molding process. During fabrication of the spacer 410, plastic flows in and about the cables 412a and 412b and thereafter sets up between the surface 457 and the surface 458 as shown in FIG. 18. If desired, prior to molding, the segments 416 and 418 may be pulled away from one another along the axis F, tensioning and if desired, expanding the portion 420 at the slit 424, followed by molding of the spacer 410 about the portion 420. Some or no tension may be placed on the cables 412. When the jig or holding mechanism is released after the molding of the spacer 410 is completed, the tensioned portion 420 will tend to draw together along the axis F, thereby placing a compressive force on the spacer 410 along the axis F with the spacer 410 keeping the portion 420 in tension. It is noted that in some embodiments of the invention, the spacer 410 is molded entirely over the plates 422 and 423 as previously described herein with respect to the assemblies 201 and 301.

The assembly 401, that may be pre-tensioned and/or pre-bent at the segment 420, is eventually positioned in an open or percutaneous manner in cooperation with the at least two bone screws 25 with the plates 422 and 423 and the spacer 410 disposed between the two bone screws 425 and the end portions 416 and 418 each within the U-shaped channels of the two bone screws 25. A closure structure 27 is then inserted into and advanced between the arms of each of the bone screws 25. The closure structure 27 is rotated, using a tool (not shown) engaged with the closure inner drive until a selected pressure is reached at which point the core 408 is locked into position within the U-shaped channel of each of the bone screws 25 as previously described herein with respect to the assemblies 1, 101, 201 and 301. For example, about 80 to about 120 inch pounds pressure may be required for fixing the bone screw shank with respect to the receiver at a desired angle of articulation.

The assembly 401 is thus substantially dynamically loaded and oriented relative to the cooperating vertebra, providing relief (e.g., shock absorption) and protected movement with respect to flexion, extension, distraction, compressive, torsion and shear forces placed on the assembly 401 and the two connected bone screws 25. The helical slit 424 and cooperating elastic spacer 410 allow the core 408 to twist or turn, providing some relief for torsional stresses. The spacer 410, however limits such torsional movement as well as bending movement, providing spinal support. Furthermore, the cables 412 provide additional support and act as a check against continued distraction of the slitted portion 420 when the plates are flexed and compressed against the spacer 410, and against additional unwanted or over-flexure of the relatively flexible slitted portion 420 and relatively flexible spacer 410. Also, when the spacer 410 is compressed during installation, the spacer 410 and slit 424 combination allow for some additional protected extension or distraction of both the core 408 and the spacer 410 as well as compression of the assembly 401.

Eventually, if the spine requires more rigid support, the connecting member assembly 401 according to the invention may be removed and replaced with another longitudinal connecting member, such as a solid rod, having the same diameter as the core member 408 end portions 416 and 418, utilizing the same bone screws 25. Alternatively, if less support is eventually required, a less rigid, more flexible assembly, for example, an assembly 1 made of a more flexible material or an assembly 401 having a slit of different pitch, but with end portions having the same diameter as the core 408 end portions 416 and 418, may replace the assembly 401, also utilizing the same bone screws 25.

With reference to FIG. 20, another alternative longitudinal connecting member assembly according to the invention, generally 501 includes an elongate core member or segment, generally 508, an outer sleeve or spacer 510 and one cable 512. The core member 508, the spacer 510 and the cable 512 are identical or substantially similar to the respective core member 408, spacer 410 and cables 412 previously described herein with respect to the assembly 401. The assembly 501 differs from the assembly 401 in that the assembly 501 has only one cable 512 and the core member 508 is bent at a dynamic mid-portion 520 having a helical slit 524 during molding of the spacer 510 about the mid-portion 520. When implanted between a pair of bone screws 25 with the cable 512 positioned at a location most posterior of the spine and the core member 508, the bent core 508 and cooperating spacer 510 provide additional support or correction to the spine, for example, when correcting spinal lordosis. Furthermore, the single posteriorly placed cable 512 acts as a check or limit on bending movement of both the core 508 and the spacer 510, as well as over distraction of the slit. In other embodiments of the invention, the plates on either side of the spacer 510 may be shaped similar to the plates 321 and 323 previously described herein with respect to the assembly 301, resulting in an axis of translation being transferred from a posterior to an anterior position (e.g., anterior of a facet joint, guarding against overload of such facet in compression).

With reference to FIG. 21, another alternative longitudinal connecting member assembly according to the invention, generally 601, includes an elongate core member or segment, generally 608, a molded outer sleeve or spacer 610 and a pair of cables 612a and 612b. The core member 608, the spacer 610 and the cables 612a and 612b are identical or substantially similar to the respective core member 408, spacer 410 and cables 412a and 412b previously described herein with respect to the assembly 401. The assembly 601 differs from the assembly 401 in that during the assembly of the cables 612a and 612b onto the integral plates of the core member 608, such cables are oriented in a criss-cross manner as compared to the parallel orientation of the cables 412a and 412b of the assembly 401. Such criss-cross orientation provides further support and limits against bending of the spacer 610 and slitted portion of the core 608. To provide the greatest support, the cables 612a and 612b are mounted at posterior locations ten o'clock and two o'clock as previously described herein with respect to the assembly 401.

It is to be understood that while certain forms of the present invention have been illustrated and described herein, it is not to be limited to the specific shapes forms or arrangements of parts described and shown.

1. In a medical implant assembly having at least two bone attachment structures cooperating with a longitudinal connecting member, the improvement wherein the longitudinal connecting member comprises:
a) an inner core having a stop and a slitted segment;
b) an outer spacer covering the slitted segment; and
c) a compression member attached to the core pressing the spacer against the stop and tensioning the slitted segment prior to implantation of the implant assembly.

2. The improvement of claim 1 wherein the slitted segment has a helical slit.

3. The improvement of claim 1 wherein the spacer is elastic.

4. The improvement of claim 1 wherein the spacer has a surface with at least one groove formed therein.

5. The improvement of claim 1 wherein the inner core has a first longitudinal section and an integral second longitudinal second, the first longitudinal section having the slit, the first longitudinal section extending between first and second bone attachment structures and the second longitudinal section extending between the second bone attachment structure and a third bone attachment structure.

6. The improvement of claim 5 wherein the second longitudinal section has a second slit.

7. The improvement of claim 5 wherein the second longitudinal section is a solid rod.

8. The improvement of claim 1 wherein the compression member is threadably mated to the inner core.

9. The improvement of claim 1 wherein the compression member further comprises a planar surface disposed adjacent the spacer.

10. The improvement of claim 1 wherein the stop is a first stop and the compression member is a second stop, the slitted segment being located between the first and second stops, the outer spacer being over-molded about the slitted segment and between the first and second stops, the outer spacer molded during at least one of tensioning and bending of the slitted segment.

11. The improvement of claim 10 wherein the outer spacer is over-molded about and surrounding the first and second stops.

12. The improvement of claim 10 further comprising a band disposed between and connecting the first and second stops.

13. The improvement of claim 12 wherein the band is an elastic band disposed about the first and second stops.

14. In a medical implant assembly having at least two bone anchors cooperating with a longitudinal connecting member, the improvement wherein the longitudinal connecting member comprises:
a) an inner core having a helical slit, the core being at least one of bent and tensioned at the helical slit;
b) a first stop plate integral with the inner core;
c) an elastic spacer surrounding the helical slit; and
d) a second stop plate, the elastic spacer substantially disposed between the first stop plate and the second stop plate.

15. The improvement of claim 14 wherein the second stop plate is advanceable along the inner core in a direction toward the elastic spacer for compressing the elastic spacer between the first stop plate and the second stop plate.

16. The improvement of claim 14 wherein the second stop plate is mounted on a ring, the ring threadably mated to the inner core, the ring tensioning the inner core and the second stop plate compressing the elastic spacer.

17. The improvement of claim 14 wherein the inner core has a first longitudinal section and an integral second longitudinal second, the first longitudinal section having the slit, the first longitudinal section extending between first and second bone attachment structures and the second longitudinal section extending between the second bone attachment structure and a third bone attachment structure.

18. The improvement of claim 17 wherein the second longitudinal section has a second slit.

19. The improvement of claim 17 wherein the second longitudinal section is a solid rod.

20. The improvement of claim 14 wherein the spacer is molded over the first and second plates.

21. The improvement of claim 20 further comprising a band surrounding a portion of the first and second plates.

22. In a medical implant assembly having at least two bone attachment structures cooperating with a longitudinal connecting member, the improvement wherein the longitudinal connecting member comprises:
a) an inner core having an axis, a pair of spaced abutment surfaces and a slitted segment disposed axially between the pair of abutment surfaces;
b) a molded elastic outer spacer spaced from the slitted segment and engaging each of the abutment surfaces; and
c) at least one cable disposed in the spacer and spanning between the abutment surfaces.

23. The improvement of claim 22 wherein the slitted segment has a helical slit.

24. The improvement of claim 22 wherein the at least one cable is a first cable and a second cable.

25. The improvement of claim 24 wherein the first and second cables are spaced from one another at about one-hundred-twenty degrees measured with respect to the axis.

26. The improvement of claim 24 wherein the first cable is oriented at a diagonal with respect to the second cable.

27. The improvement of claim 22 wherein the slitted segment is bent.

28. The improvement of claim 22 wherein the slitted segment is in tension.

29. The improvement of claim 22 wherein the slitted segment is expanded during molding of the spacer thereabout.

30. The improvement of claim 22 wherein the cable is elastic.

31. In a medical implant assembly having at least two bone anchors cooperating with a longitudinal connecting member, the improvement wherein the longitudinal connecting member comprises:
a) an inner core having a helical slit;
b) at least one stop plate integral with the inner core; and
c) an over-molded elastic spacer surrounding the helical slit, the at least one stop plate and the spacer each extending in at least one direction lateral to the core an amount sufficient for the stop plate and the spacer to cooperate to substantially resist bending moment of the core.

32. The improvement of claim 31 wherein the stop plate is a first stop plate and further comprising a second stop plate, the elastic spacer substantially disposed between the first stop plate and the second stop plate.

33. The improvement of claim 32 wherein the spacer is molded over the first and second stop plates.

34. The improvement of claim 32 wherein the first and second stop plates are elongate in an anterior operational direction.

35. The improvement of claim 32 further comprising a band attached to a portion of each of the first and second stop plates.

36. The improvement of claim 35 wherein the band surrounds each of the first and second plates.
